(19) United States
(12) Patent Application Publication
(10) Pub. No.: US 2021/0137039 A1
(43) Pub. Date: May 13, 2021

(54) SMART POLLINATION SYSTEM

(71) Applicant: SANDEEP KUMAR CHINTALA, GREENFORD (GB)

(72) Inventor: SANDEEP KUMAR CHINTALA, GREENFORD (GB)

(21) Appl. No.: 16/969,546

(22) PCT Filed: Feb. 13, 2019
(86) PCT No.: PCT/GB2019/050378

§ 371 Date: Aug. 12, 2020

(30) Foreign Application Priority Data
Feb. 13, 2018 (IN) 201841005490

(51) Int. Cl. A01H 1/02

(52) U.S. Cl. CPC A01H 1/027

(57) ABSTRACT

A smart pollination system includes a smart pollination apparatus (100) that is machine learned and uses artificial intelligence engine for pollination. The smart pollination apparatus (100) is communicatively coupled to a global communications system (GCS). The GCS and the smart pollination apparatus (100) manage the pollination trends with the help of artificial intelligence and machine learning.

### BACKGROUND

### 1. Technical Field

The subject matter described herein, in general, relates to pollination optimization, and in particular relates to a smart pollination system for optimization of pollination using machine learning and artificial intelligence.

### 2. Related Art

Plants are known to reproduce via pollination. The pollination is a process, in which a genetic material, such as a pollen, is transferred amongst plants, or flowers of plants, or within a flower by itself. In general, the pollination may be managed by manually transporting pollen, or by raising bees that pollinate plants. The bees act as carriers of pollen. Other insects or animals also act as carriers of pollen.

In general, the process of pollination in plants is carried out by bees. The bees pollinate plants by acting as a carrier of a genetic material, i.e. pollens. The pollen is transferred from male reproductive organs of a plant to the female reproductive organs of a plant, thereby enabling fertilization within the plant.

The global crop pollination by bees has a great economic significance. Bees are a vitally important part of the ecosystem and responsible for pollinating many of the plants and crops that humans rely on to live. Bees are important contributors to world food production and nutritional security. However, the bees number are in decline and resulting in reduced global crop pollination. Use of pesticides on plants may be one of the factors involved in reducing the bees number.

Another factor for global crop pollination reduction may be inbreeding depression within plants. In one example, the inbreeding depression may be especially applicable to papaya and other similar plants. The inbreeding depression is the reduced biological fitness in a given population as a result of inbreeding, or breeding of related individuals and ultimately resulting in reduced global crop pollination.

Though the decline in number of the bees is an important and major factor resulting in reduced global crop pollination, the efficiency of natural pollinations such as bees has always been an unknown factor. There could be lot of wastage of pollens during pollination by bees, because there is no control over the bees on how they pollinate and how many pollens are effectively utilized during pollination.

### SUMMARY

To this end, a smart pollination system is proposed, which is used for an efficient artificial pollination of plants. The smart pollination system is a smart pollination apparatus.

In an implementation of the present subject matter, the smart pollination apparatus is machine learned and uses artificial intelligence for the artificial pollination. The smart pollination apparatus is a flying unit. In one example, the smart pollination apparatus is communicatively coupled to a central network system. In one example, the central network system may be a global communications system (GCS). The GCS manages the pollination trends with the help of an artificial intelligence (AI) engine and notifies nearby smart pollination apparatus upon identifying an ideal condition to initiate pollination.

In operation, the smart pollination apparatus flies to a target host upon identifying the ideal condition to initiate the artificial pollination. The identification of the ideal condition is based on machine learning and artificial intelligence. Upon reaching the target host, pollens are collected from the target host by the smart pollination apparatus and further stores the pollens collected from the target host. Further, a recipient near the target host is identified based on machine learning and artificial intelligence and, upon identifying the recipient, the smart pollination apparatus flies to the identified recipient for depositing the stored pollens into the identified recipient in a predetermined quantity. The deposition of pollen may be done by, but is not limited to, spraying, injecting, etc., depending on the suitability for the identified recipient. Upon depositing the predetermined quantity of the pollens to the recipient and determining that there is no other recipient in the vicinity, the surplus pollens may be taken to a storage warehouse as per instructions received from the GCS. The smart pollination apparatus is automatically controlled.

In the ideal condition, the GCS and the smart pollination apparatus rely on the extensive knowledge base of hosts, recipients, weather, seasonal trends, air quality, soil quality and other similar suitable conditions required for pollination that's been built up either guides the smart pollination apparatus to a nearby suitable recipient capable of accepting the collected pollens or as part of the active learning process, the smart pollination apparatus finds a suitable recipient in the flight path while actively scanning the landscape and shares this information to GCS and based on the AI engine validating the information gathered, the GCS may instruct the smart pollination apparatus to deposit stored pollens to the recipient.

In one implementation of the present subject matter, if the recipient is identified, the smart pollination apparatus is instructed not to deposit the stored pollen because of other influencing factors such as bad weather forecasted, poor soil conditions, excess pesticides detected, potential war or hostile activity taking place or threats predicted in such area. In such cases, the stored pollen is taken to the warehouse and stored until a suitable recipient is identified. In an example, the stored pollens are transported to another geographic location where a suitable recipient is available.

The subject matter described in the previous embodiments can be embodied in many ways as would be obvious to a person skilled in the art. For example, the components of the smart pollination apparatus can be made in different shapes and sizes.

With the above disclosed implementations of the present subject matter, the pollination can be optimized by accurately determining how, where, when and how much pollens are required to be pollinated. The smart pollination apparatus enables the pollination process to take place at the most fertile stages of the plant, thus maximizing and increasing the quality of the yield. The smart pollination apparatus of the present subject matter provides a fully autonomous solution that optimizes the pollination process and increases the yield by manifold.

The smart pollination apparatus of the present subject matter may be used not only as an alternative for the bees but also for other insects and animals that carry out the pollination process. In one example, smart pollination apparatus of the present subject matter carries out abiotic pollination.

### THE DRAWINGS

These and other advantages of the present subject matter will be described in a greater detail in conjunction with the following FIGS. 1-3 and description. The manner in which the smart pollination apparatus is implemented and used shall be explained in detail with respect to the FIGS. 1-3, wherein:

FIG. 1 shows a block diagram of a smart pollination apparatus, as per an implementation of the present subject matter;

FIG. 2 shows a block diagram of a smart pollination apparatus in communication with a central network system, as per an implementation of the present subject matter;

FIG. 3 shows a flow diagram for a method of operation of a smart pollination apparatus, as per an implementation of the present subject matter.

### DETAILED DESCRIPTION

It should be noted that the description merely illustrates the principles of the present subject matter. It will thus be appreciated that those skilled in the art will be able to devise various arrangements that, although not explicitly described herein, embody the principles of the present subject matter and are included within its scope. Furthermore, all examples recited herein are intended only to aid the reader in understanding the principles of the present subject matter. Moreover, all statements herein reciting principles, aspects and implementations of the present subject matter, as well as specific examples thereof, are intended to encompass equivalents thereof.

FIG. 1 shows a block diagram of a smart pollination apparatus 100 for carrying out an artificial pollination, as per an implementation of the present subject matter. The smart pollination apparatus 100 uses artificial intelligence (AI) engine and machine learning for optimized pollination. The smart pollination apparatus 100 is a flying unit. In one example, the flying unit is an unmanned aerial vehicle (UAV). The smart pollination apparatus 100 is communicatively coupled with a central network system 200, which is shown in FIG. 2. In one example, the central network system 200 may be a global communications system (GCS).

Returning to FIG. 1, the smart pollination apparatus 100 uses artificial intelligence (AI) engine and machine learning for identifying a target host. The target host is a host plant. In one example, the identification of the target host is carried out using imagery mapping techniques.

The smart pollination apparatus 100 includes a suction unit 102 for collecting pollens from the target host. The pollens may refer to pollen grains. In one example, the smart pollination apparatus 100 includes a combination of suction units 102 for collecting or harvesting pollens from the target host. In one example, the suction unit 102 includes an aspiration pump for collecting pollens from the target host.

The smart pollination apparatus 100 includes a storage unit 104 for securely storing the pollens being collected or harvested from the host plant at prespecified controlled conditions to maintain properties of the collected pollens. The storage unit 104 is connected to the suction unit 102. In one example, a plurality of storage units 104 is provided that can securely store the pollens being collected or harvested from the host plant. The storage unit 104 is a contamination free storage unit. In one example, the storage unit 104 may be connected to a weighing unit and a profiling unit. The pollens collected from the host plants may be weighed and profiled for their types with the relevant data shared with the central network system 200.

The smart pollination apparatus 100 include an AI engine, which is programmed to eject pollens if a suitable recipient is identified for the stored pollens on the flight path of the smart pollination apparatus 100. In another implementation of the present subject matter, the smart pollination apparatus 100 can return to a warehouse where the pollens may be securely stored and maintained under ideal environmental conditions that is necessary for the pollens depending on the profile of the pollens. The storage unit 104 of the smart pollination apparatus 100 is detachable. The storage unit 104 can be detached and used as storage containers within the warehouse. Each warehouse can have variable temperature/environment-controlled zones, thus creating optimum conditions to store different type of pollens within the same warehouse.

In yet another implementation of the present subject matter, the stored pollens from the warehouse can also transported to other geographically located warehouses by rail, road, water or air. Upon running the necessary analytics, the stored pollens can be pollinated by the smart pollination apparatus 100 or by Structured Wireless-Aware Network (swam) network of the smart pollination apparatus 100 upon identifying the right recipient and environmental conditions. Examples of environmental conditions may include, but is not limited to, favorable seasons, geographic locations and other factors that could result in better yield and quality of crop produce.

Further, the smart pollination apparatus 100 includes a spray unit 106 connected to the storage unit 104. In one example, the smart pollination apparatus 100 may include a combination of spray units 108. In one example, the smart pollination apparatus 100 includes a combination of suction units that can be converted into a spray unit when necessary. In one example, the spray unit 106 may be used for depositing the stored pollens over a recipient having multiple female reproductive parts. In one example, the spray unit 106 may be used for depositing pollens over a plurality of recipients. The necessary analytics of the recipient are monitored by the smart pollination apparatus 100 and, based on the monitoring of the necessary analytics of the recipient, the pollens can be sprayed over the recipient injected into the recipient using higher accuracy, thus resulting in better yield as compared to natural pollination process.

In another implementation of the present subject matter, the smart pollination apparatus 100 includes an inserter 108, connected to the storage unit 104 for precisely inserting pollens into the recipient using Machine Learning and Artificial Intelligence. In one example, the inserter 108 may be a micro-peeler. The necessary analytics of the recipient are monitored by the smart pollination apparatus 100 to identify the exact location of the female reproductive part and, based on the monitoring of the necessary analytics of the recipient, the pollens can be precisely inserted into the female reproductive part of the recipient by the inserter 108, thus resulting in better yield as compared to natural pollination process.

Using Machine Learning and Artificial Intelligence, the fertility and applicable surface area of the recipient may be determined and the appropriate quantity of pollens may be ejected by the smart pollination apparatus 100. The ejection of the pollens will be automatically triggered by the smart pollination apparatus 100 using machine learning and artificial intelligence. In one example, the ejection of the pollens may be carried out by a remote source in communication with the smart pollination apparatus 100. In another example, the smart pollination apparatus 100 provides options for manual override where the smart pollination apparatus 100 can be manually controlled in emergency situations.

Further, the smart pollination apparatus 100 includes robotic arms 110. Upon identifying the target host by the smart pollination apparatus 100 or upon receiving information from the GCS about the target host, the smart pollination apparatus 100 flies to the target host and then the robotic arms 110 latch onto the target host and cause vibrations for shedding the pollens within the target host. The pollens are then collected into the storage unit 104.

The smart pollination apparatus 100 includes a plurality of cameras 112 for capturing patterns on flowers of plants for identifying the target host and the recipient based on the captured patterns. The cameras 112 may include, but is not limited to, Ultra High Definition cameras, thermal, night vision cameras, x-ray vision cameras, image recognition cameras, infrared and ultraviolet cameras and the like. The ultraviolet cameras can perfectly capture the patterns on a flower to find nectar and pollen in a same manner the bee does.

In an implementation of the present subject matter, the smart pollination apparatus 100 includes a power supply 116. In one example, the power supply 116 may be wireless power supply. The wireless power supply unit has over the air capabilities via magnetic, radio, ultrasound, acoustics, Light Fidelity or any other means. In another example, the power supply 116 may be perpetual energy systems or swappable battery or energy storage systems to provide power to flying unit 102. In yet another example, the power supply 116 may be solar or photovoltaic system to power the flying unit 102.

Automated wireless power charging network towers or stations can be strategically placed in various geographic locations across the planet and/or a network of satellites capable of supplying wireless power to the target smart pollination apparatus 100 while in flight to power the smart pollination apparatus 100 can be placed in a geo stationary orbit. Wireless Power charging protocol can be initiated at any time when the GCS identifies that the smart pollination apparatus 100 power levels are dropping below a certain threshold or the smart pollination apparatus 100 requests additional power supply in order to extend the flight duration. In another example, a power bank flying unit may travel within a cluster of smart pollination apparatuses 100 and supply power wirelessly to any smart pollination apparatus 100 within the cluster when necessary. Multiple power bank flying units can also be located at various operational bases to support cluster of smart pollination apparatuses 100 carrying out pollination, as and when necessary. The power bank flying units can also receive wireless power from the automated wireless power charging network towers or stations. In one example, the power bank flying units can receive power from the satellite.

In another implementation of the present subject matter, automated battery or power unit swapping stations can be strategically placed in various geographic locations across the planet to enable continuity of operations. When the power levels of the smart pollination apparatus 100 is falling below a certain threshold, the AI engine within the smart pollination apparatus 100 or the GCS can guide the smart pollination apparatus 100 to the nearest swap charging station. Further, the smart pollination apparatus 100 is fully charged once the battery or power storage unit is swapped. The power bank flying units can also have the capabilities to act as Swap Charge Stations and battery or power storage units can be swapped automatically between the pollenating flying units and Power Bank flying units while in flight. All communications between the flying units, Wireless Power stations or towers or satellites and the GCS is monitored, stored and analyzed by the GCS.

In one example, the smart pollination apparatus 100 can also run on hydrogen cells, diesel, petrol, nuclear or any other type of fuel.

The smart pollination apparatus 100 may include other components 118 which are required for optimized pollination. Other components may include, but is not limited to, a spectrometer, minerals and compound analyser and the like.

FIG. 3 shows a flow diagram for a method for operation 300 of a smart pollination apparatus 100, as per an implementation of the present subject matter. The smart pollination apparatus 100 may be communicatively coupled to the GCS. Both the smart pollination apparatus 100 and the GCS utilize artificial intelligence and machine learning for optimizing the pollination process.

In operation, the smart pollination apparatus 100 may identify a target host (step 302) and a suitable recipient near the target host for pollination using imagery mapping techniques. Upon identifying the target host, the smart pollination apparatus 100 flies near the identified target host and the robotic arms 110 of the smart pollination apparatus 100 latch onto the identified target host and cause vibrations that results in the shedding of pollens within the target host. The robotic arm 110 of the smart pollination apparatus 100 is guided by machine learning and artificial intelligence for ensuring that the right amount of vibrations induced to the target host, where necessary, to enable release of pollens within the target host.

The pollens are then collected (step 304) by the suction unit 102 and further being stored (step 306) in the storage unit 104. Further, the stored pollens are examined by the smart pollination apparatus 100 for identifying how mature the pollens are. In an example, if a pollen is in an early stage, then the pollen can be sent to storage and if a pollen is relatively mature then a suitable recipient is required to be identified. The examination of pollens can be carried out even for other mode of pollen collection such as a suction mechanism by a robotic arm of the smart pollination apparatus and is not limited to a robotic arm 110 from the smart pollination apparatus 100 latching onto the host male part of the plant. In one example, the robotic arm 100 has an ability to move around the smart pollination apparatus 100 via tracks or electromagnetic surface on the smart pollination apparatus 100, giving the robotic arm 110 maximum freedom for mobility to carry out all the operations of the smart pollination apparatus 100.

Further, the suitable recipient may be identified (step 308) using cameras 112 based on artificial intelligence and machine learning for pollination. Upon identifying the suitable recipient, either the spray unit 106 may spray the pollens onto the identified recipient (step 310) or the inserter 108 precisely inserts the pollens onto the identified recipient.

In one example, a global geographic landscape may be created either by the smart pollination apparatus 100 or the GCS 200 to identify the hosts and recipients, managing seasonal yield charts, pollen quality and quality volumetric, weather management, regional air quality and also potentially soil quality and other factors that may have an influence on the pollination process.

Further, smart pollination apparatus 100 includes obstacle avoidance functions, real time communications within immediate the swam network, extended swam networks, headquarters and other globally positioned communication bases, air traffic controllers and other vital services.

In one implementation of the present subject matter, features of the smart pollination apparatus 100 include power management, real time flying health analytics, manage remote software updates, report to base in case of the the smart pollination apparatus 100 becoming non-functional or ability to track the smart pollination apparatus 100 when it goes offline using a secondary or backup communications system.

The functions of the artificial intelligence and machine learning may evolve over time and new feature can be added to the smart pollination apparatus 100 as and when required.

The smart pollination apparatus 100 using the machine learning utilizes most of the suitable data from previous research for optimizing pollination process. The smart pollination apparatus 100 using the machine learning maximizes the pollen harvesting from hosts and ejection/insertion process into the recipients to maximize quantity and quality yield. Different type of hosts and recipients may require difference process or combination of difference processes to carry out the pollination actions. The smart pollination apparatus 100 using the machine learning optimize operational activity, such as but not limited to flight motions, power management, storage management, swam formations, and obstacle avoidance measures. In one example, on a regular basis, the smart pollination apparatus 100 carries out standalone activities where the smart pollination apparatus 100 uses the cameras 112 and record how bees or other pollen transporters such as butterflies, ants etc. harvest the pollen from hosts and how they deposit the harvested pollen into a recipient. Further, the recorded data is feed into the GCS and send updates to improve the operations with the smart pollination apparatus 100.

In another implementation of the present subject matter, the GCS 200, whether on the cloud or data centers or connected operational bases, manages the entire pollination activity across the planet using artificial intelligence. All smart pollination apparatuses 100 may have call signed or identifications and all communications between the GCS 200 and the smart pollination apparatuses 100 are encrypted and secure. The GCS 200 continuously monitors the whereabouts, power supply/battery and smart pollination apparatuses 100 health at all times.

If the standard operational functionality of the smart pollination apparatus 100 goes offline, then a standby offline smart pollination apparatus 100 tracker protocol may be initiated using a low power backup system in the smart pollination apparatus 100. An alert signal may be sent back to the GCS and a recovery system may be initiated to recover the offline/non-operational flying unit. If the recovery flying unit is unable to track or recover the offline/non-operational smart pollination apparatus 100, in such a case human intervention may be required for recovering the offline/non-operational smart pollination apparatus 100.

In an implementation of the present subject matter, when the hosts and the recipients to be pollinated are below ground or under water. In such cases, water proof smart pollination apparatus 100 may be sent to carry out the pollination operations.

In another implementation of the present subject matter, if a pollen can self-pollinate without a need for live recipient then the smart pollination apparatus 100 may identify suitable soil (or other applicable method) to plant such a pollen. All this knowledge will be continuously updated in the GCS.

In yet another implementation of the present subject matter, Geo-mapping smart pollination apparatus 100 may scan the earth to identify and map all the different hosts and/or recipients in the flight path, creating an extensive knowledge base for the operational smart pollination apparatuses 100. In one example, information from third party sources can also be used to enrich the data relating to pollination.

In an implementation of the present subject matter, the smart pollination apparatus 100 may build deeper individual plant level 360-degree 3D mapping data and identifying where the male and female parts of the plant are and understanding their fertility cycle and accordingly deliver better quality and quantity of yield.

In an implementation of the present subject matter, a transporter drone may be deployed that houses and transports various smart pollination apparatuses 100, and also that can act as refueling station especially with the swappable battery technology for the pollination operation drones, or one carrying sufficient energy storage capacity and capable of wirelessly powering pollination drones while they are in operation when the power levels of the operation drones drop below a certain threshold.

The identification of the target host and recipient by using machine learning and artificial intelligence include olfactory sensing and bioassays for ionic liquid gels to replicate the character of the smart pollination apparatus 100 similar to a natural pollinator.

Although implementations for the smart pollination apparatus 100 are described, it is to be understood that the present subject matter is not necessarily limited to the specific features described. Rather, the specific features are disclosed as implementations.

1. A smart pollination apparatus (100) for carrying out an artificial pollination, wherein the smart pollination apparatus (100) is machine learned and uses artificial intelligence for the artificial pollination, wherein the smart pollination apparatus (100) is to:
fly to a target host upon identifying an ideal condition to initiate the artificial pollination, wherein identification of the ideal condition is based on machine learning and artificial intelligence;
collect pollens from the target host;
store the pollens collected from the target host;
identify a recipient near the target host based on machine learning and artificial intelligence; and
upon identifying the recipient, fly to the identified recipient for depositing the stored pollens into the identified recipient in a predetermined quantity.

2. The smart pollination apparatus (100) as claimed in claim 1, wherein the smart pollination apparatus (100) comprises a robotic arm (110) for latching onto the target host and causing vibrations for shedding the pollens within the target host.

3. The smart pollination apparatus (100) as claimed in claim 1, wherein the smart pollination apparatus (100) comprises a suction unit (102) for collecting pollens from the target host.

4. The smart pollination apparatus (100) as claimed in claim 3, wherein the suction unit (102) comprises an aspiration pump for collecting the pollens from the host plant.

5. The smart pollination apparatus (100) as claimed in claim 3, wherein the smart pollination apparatus (100) comprises a storage unit (104), connected to the suction unit (102), for storing the collected pollens at prespecified controlled conditions to maintain properties of the collected pollens.

6. The smart pollination apparatus (100) as claimed in claim 5, wherein the storage unit (104) is detachable.

7. The smart pollination apparatus (100) as claimed in claim 6, wherein the storage unit (104) comprises a profiling unit for profiling types of the pollens stored within the storage unit (104).

8. The smart pollination apparatus (100) as claimed in claim 5, wherein the smart pollination apparatus (100) comprises an inserter (108), connected to the storage unit (104), for precisely inserting the stored pollens into the identified recipient.

9. The smart pollination apparatus (100) as claimed in claim 5, wherein the smart pollination apparatus (100) comprises a spray unit (106), connected to the storage unit (104), for spraying the stored pollens over the identified recipient with multiple female reproductive parts.

10. The smart pollination apparatus (100) as claimed in claim 1, wherein the smart pollination apparatus (100) creates a global geographic landscape based on prespecified parameters for identifying the target host and the recipient.

11. The smart pollination apparatus (100) as claimed in claim 1, wherein the smart pollination apparatus (100) comprises a plurality of cameras (112) for capturing patterns on flowers of plants for identifying the target host and the recipient based on the captured patterns.

12. The smart pollination apparatus (100) as claimed in claim 1, wherein the smart pollination apparatus (100) is an unmanned aerial vehicle.

13. The smart pollination apparatus (100) as claimed in claim 1, wherein the smart pollination apparatus (100) is controlled by a remote source in communication with the smart pollination apparatus (100).

14. The smart pollination apparatus (100) as claimed in claim 1, wherein the smart pollination apparatus (100) is automatically controlled.

15. The smart pollination apparatus (100) as claimed in claim 1, wherein smart pollination apparatus (100) comprises a power supply (114) to power the smart pollination apparatus (100).

16. The smart pollination apparatus (100) as claimed in claim 15, wherein the power supply (114) is a wireless power supply.

17. A method for carrying out an artificial pollination using a smart pollination apparatus (100) as claimed in claim 1.
